(19) United States
(12) Patent Application Publication
(10) Pub. No.: US 2019/0111208 A1
(43) Pub. Date: Apr. 18, 2019

(54) INFUSION APPARATUS

(71) Applicants: Inventec Appliances (Pudong) Corporation, Shanghai (CN); Inventec Appliances Corp., New Taipei City (TW); Inventec Appliances (Shanghai) Co., Ltd., Shanghai (CN)

(72) Inventors: Yi-Yu LEE, New Taipei City (TW); Wen-Tso TSENG, New Taipei City (TW); Shou-Tien YIN, New Taipei City (TW)

(21) Appl. No.: 15/822,200

(22) Filed: Nov. 26, 2017

(30) Foreign Application Priority Data
Oct. 12, 2017 (CN) 201710964932.1

(51) Int. Cl. A61M 5/168; A61M 5/142; A61M 39/10

(52) U.S. Cl. CPC A61M 5/16854; A61M 5/142; A61M 2005/16863; A61M 5/14228; A61M 2205/3331; A61M 39/10

(57) ABSTRACT

An infusion apparatus includes a housing, a door assembly, an infusion tube assembly, a pressing mechanism, and a QTC (Quantum tunnelling composite) pressure sensor. The housing has an opening. The door assembly is connected to the housing and configured to cover the opening. The infusion tube assembly has a first tube located in the housing. The pressing mechanism is disposed in the housing and configured to press the first tube. When the door assembly covers the opening, the QTC pressure sensor is located between the housing and the door assembly and configured to detect a pressing operation of the pressing mechanism to the first tube.

### RELATED APPLICATIONS

This application claims priority to China Application Serial Number 201710964932.1, filed Oct. 12, 2017, which is herein incorporated by reference.

### BACKGROUND

### Technical Field

The present disclosure relates to an infusion apparatus.

### Description of Related Art

At present, infusion apparatuses in the clinical use can generally perform two methods of drug administration such as basic administration and manual administration. The basic administration refers to setting the time and the dose in an infusion apparatus, and a predetermined amount of drug administration is then given at the set time with an average rate. The manual administration refers to performing a one-time set of a predetermined amount of drug administration when a corresponding button of the infusion apparatus is triggered by the medical staff, patients, family members, etc.

However, the conventional infusion apparatuses often need a lot of sensors and mechanical devices to achieve purposes of detecting pressure values and pressure locations. As a result, not only the mechanisms and the assembling processes of the conventional infusion apparatuses are complex, the volume and costs are also hard to be reduced.

Accordingly, how to provide an infusion apparatus to solve the aforementioned problems becomes an important issue to be solved by those in the industry.

### SUMMARY

An aspect of the disclosure is to provide an infusion apparatus which can simplify the complexities of mechanisms and assembling processes and reduce the overall volume and costs.

According to an embodiment of the disclosure, an infusion apparatus includes a housing, a door assembly, an infusion tube assembly, a pressing mechanism, and a QTC (Quantum Tunneling Composite) pressure sensor. The housing has an opening. The door assembly is connected to the housing and configured to cover the opening. The infusion tube assembly has a first tube located in the housing. The pressing mechanism is disposed in the housing and configured to press the first tube. When the door assembly covers the opening, the QTC pressure sensor is located between the housing and the door assembly and configured to detect a pressing operation of the pressing mechanism applied to the first tube.

In an embodiment of the disclosure, the pressing mechanism is configured to press the first tube towards the door assembly.

In an embodiment of the disclosure, the QTC pressure sensor is disposed between the pressing mechanism and the first tube.

In an embodiment of the disclosure, the infusion apparatus further includes a wrapping member wrapping at an exterior of the QTC pressure sensor.

In an embodiment of the disclosure, the QTC pressure sensor is disposed at the door assembly.

In an embodiment of the disclosure, the door assembly includes an outer board and an inner board. The inner board is connected to the outer board. The QTC pressure sensor is disposed at the inner board.

In an embodiment of the disclosure, the QTC pressure sensor is embedded in the inner board.

In an embodiment of the disclosure, a hardness of the inner board is smaller than a hardness of the outer board.

In an embodiment of the disclosure, the door assembly further includes a cushioning member. The inner board is connected to the outer board through the cushioning member.

In an embodiment of the disclosure, the infusion tube assembly further includes a fixture detachably connected to the housing and configured to clamp an end of the first tube. When the door assembly covers the opening, the door assembly abuts against the fixture, and the QTC pressure sensor is further configured to detect an abutting operation of the door assembly applied to the fixture.

In an embodiment of the disclosure, the infusion tube assembly further includes a second tube. The fixture is further configured to clamp an end of the second tube, so as to connect the first tube to the second tube.

In an embodiment of the disclosure, the pressing mechanism is a peristalsis pump.

Accordingly, the infusion apparatus of the present disclosure use the QTC pressure sensor to precisely detect the exact two-dimensional position and force value of the point of pressing performed to the infusion tube assembly by the pressing mechanism, so as to become a more accurate closed loop. Therefore, the infusion apparatus of the present disclosure can replace the components known to detect the position of force point (e.g., encoders) and the components known to detect force values (e.g., pressure sensors), so as to achieve the purposes of simplifying the complexities of mechanisms and assembling processes of the infusion apparatus and reduce the overall volume and costs.

It is to be understood that both the foregoing general description and the following detailed description are by examples, and are intended to provide further explanation of the disclosure as claimed.

### BRIEF DESCRIPTION OF THE DRAWINGS

The disclosure can be more fully understood by reading the following detailed description of the embodiment, with reference made to the accompanying drawings as follows:

FIG. 1 is a perspective view of an infusion apparatus according to an embodiment of the disclosure, in which a door assembly is opened to expose an opening of a housing;

FIG. 2A is a perspective view of certain components of the infusion apparatus shown in FIG. 1, in which an inner board of the door assembly is in an open state;

FIG. 2B is another perspective view of the certain components of the infusion apparatus shown in FIG. 1, in which the inner board of the door assembly is in a closed state;

FIG. 3 is a top view of certain components of the infusion apparatus shown in FIG. 1, in which the door assembly is in a closed state;

FIG. 4 is a top view of certain components of an infusion apparatus according to another embodiment of the disclosure, in which a door assembly is in a closed state; and

FIG. 5 is a front view of certain components of an infusion apparatus according to an embodiment of the disclosure.

### DETAILED DESCRIPTION

Reference will now be made in detail to the present embodiments of the disclosure, examples of which are illustrated in the accompanying drawings. Wherever possible, the same reference numbers are used in the drawings and the description to refer to the same or like parts.

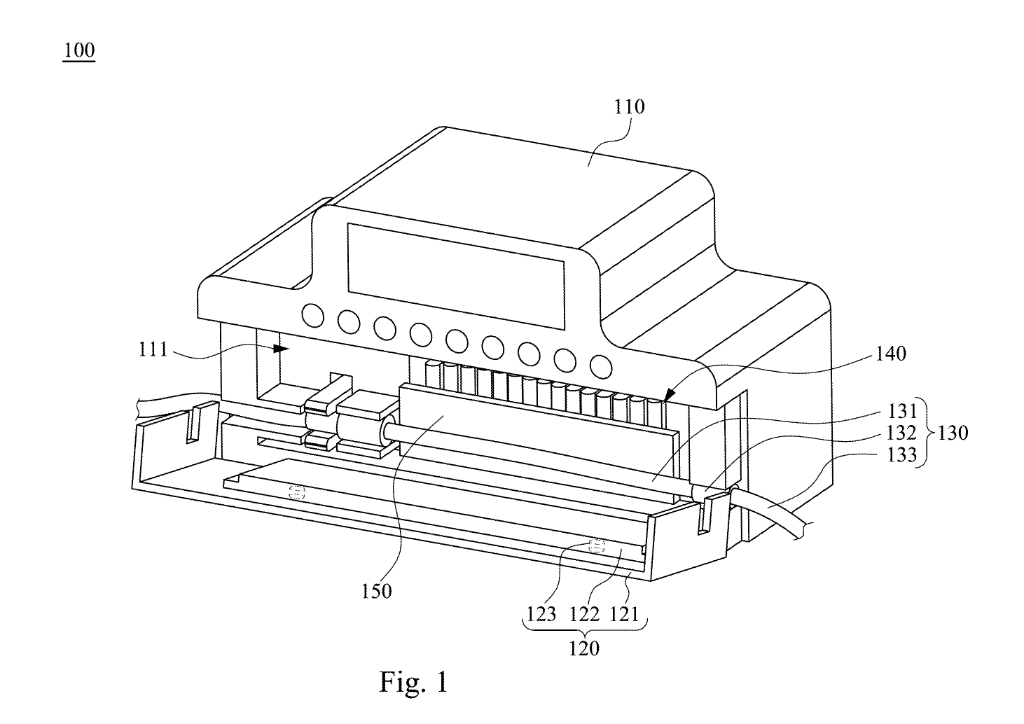

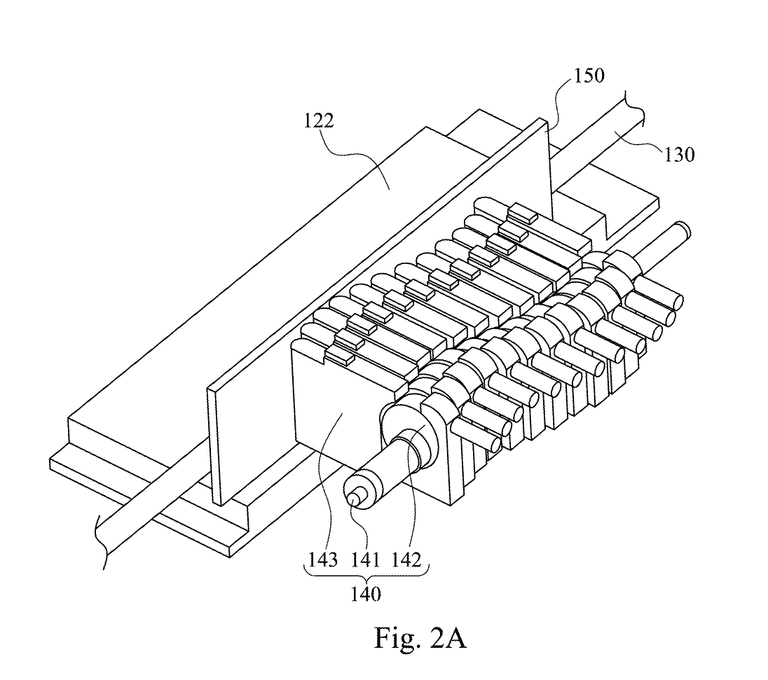

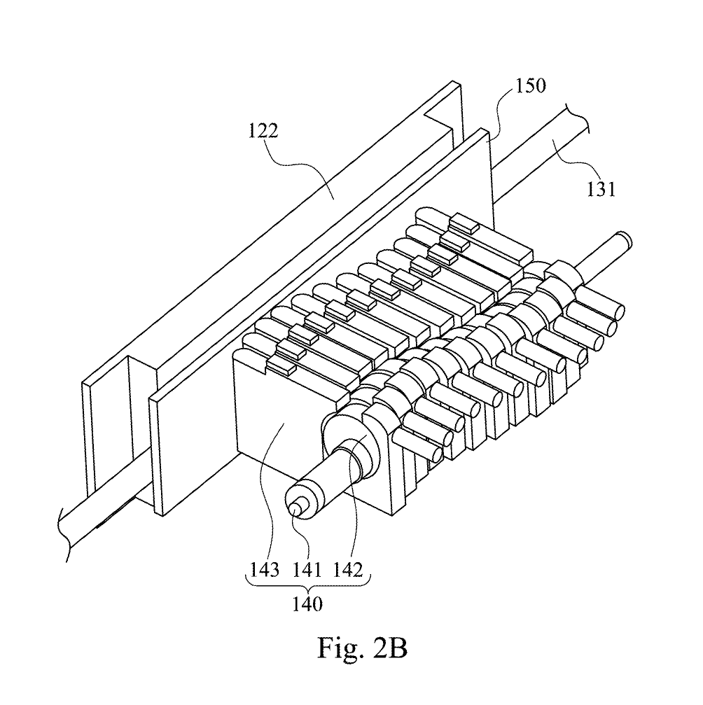

Reference is made to FIGS. 1-2B. FIG. 1 is a perspective view of an infusion apparatus 100 according to an embodiment of the disclosure, in which a door assembly 120 is opened to expose an opening 111 of a housing 110. FIG. 2A is a perspective view of certain components of the infusion apparatus 100 shown in FIG. 1, in which an inner board 122 of the door assembly 120 is in an open state. FIG. 2B is another perspective view of the certain components of the infusion apparatus 100 shown in FIG. 1, in which the inner board 122 of the door assembly 120 is in a closed state.

As shown in FIGS. 1-2B, in the present embodiment, the infusion apparatus 100 includes the housing 110, the door assembly 120, an infusion tube assembly 130, a pressing mechanism, and a QTC (Quantum Tunneling Composite) pressure sensor 150. The housing 110 has the opening 111. The door assembly 120 is connected to the housing 110 and configured to open the opening 111 (referring to FIG. 2A) and cover the opening 111 (referring to FIG. 2B). The infusion tube assembly 130 has a first tube 131 located in the housing 110. The pressing mechanism is disposed in the housing 110 and configured to press the first tube 131.

In the present embodiment, the pressing mechanism is a peristalsis pump 140 for example, but the disclosure is not limited in this regard. In practical applications, any mechanism that can sequentially press different portions of the first tube 131 of the infusion tube assembly 130 to achieve the purpose of making fluid in the first tube 131 flow can serve as the pressing mechanism of the infusion apparatus 100 of the present disclosure.

Specifically, the peristalsis pump 140 includes a shaft 141, a plurality of cam pieces 142, and a plurality of fingers 143. The shaft 141 is pivotally connected in the housing 110. The cam pieces 142 are sequentially and eccentrically sleeved onto the shaft 141, and an eccentric angle difference of any adjacent two of the cam pieces 142 is a constant value. The fingers 143 are rotatably sleeved onto the cam pieces 142 respectively and slidably disposed in the housing 110. The shaft 141 drives all of the cam pieces 142 to rotate while rotating. The housing 110 can be equipped with a guide structure (not shown) therein, so that each of the fingers 143 can reciprocally move towards and away from the opening 111 of the housing 110 while being moved by a corresponding one of the cam pieces 142. Therefore, when the door assembly 120 covers the opening 111 of the housing 110 (referring to FIG. 2B), the peristalsis pump 140 can sequentially press different portions of the first tube 131 of the infusion tube assembly 130 by the fingers 143, so as to achieve the purpose of making the fluid in the first tube 131 flow.

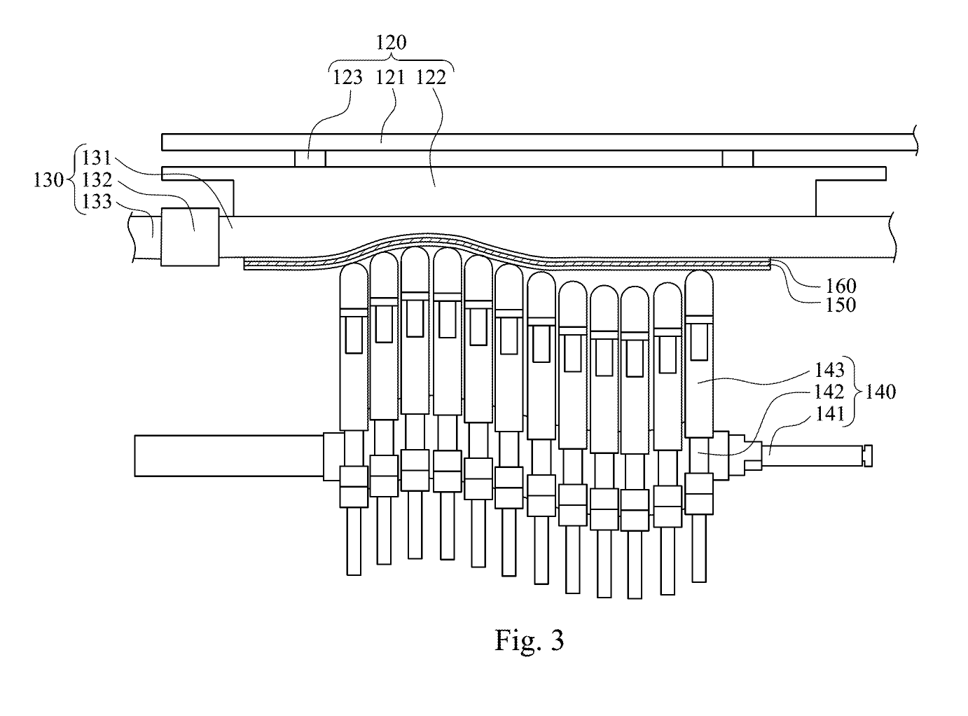

Reference is made to FIG. 3. FIG. 3 is a top view of certain components of the infusion apparatus 100 shown in FIG. 1, in which the door assembly 120 is in a closed state. As shown in FIGS. 2B and 3, in the present embodiment, the QTC pressure sensor 150 is disposed between the peristalsis pump 140 and the first tube 131. When the door assembly 120 covers the opening 111 of the housing 110, the QTC pressure sensor 150 is located between the housing 110 and the door assembly 120 and configured to detect a pressing operation of the fingers 143 of the peristalsis pump 140 applied to the first tube 131.

In detail, the QTC pressure sensor 150 is manufactured by sandwiching a composition composed of a layer of conductive particles between two layers of a glass-like material. When there is no force applied to the composition, the composition is an excellent insulator. When the composition is squeezed, stretched, or twisted, the composition becomes a metal-like conductor; and after the force is removed, the composition will return to the insulation state. In addition, when the upper layer of the glass-like material is pressed with a finger, the particles in the composition also have a characteristic of conducting a current proportionally according to the magnitude of the applied pressure. Therefore, the magnitude of the conducted current can be used to estimate the force value received by the QTC pressure sensor 150.

As a result, the QTC pressure sensor 150 can precisely detect the exact two-dimensional position and the force value of the point of pressing performed to the infusion tube assembly 130 by the fingers 143 of the peristalsis pump 140, so as to become a more accurate closed loop. Therefore, the infusion apparatus 100 of the present disclosure can replace the components known to detect the position of force point (e.g., encoders) and the components known to detect force values (e.g., pressure sensors), so as to achieve the purposes of simplifying the complexities of mechanisms and assembling processes of the infusion apparatus 100 and reduce the overall volume and costs.

As shown in FIG. 3, in the present embodiment, the infusion apparatus 100 further includes a wrapping member 160 (omitted in FIGS. 1-2B). The wrapping member 160 wraps at an exterior of the QTC pressure sensor 150 to serve as a cushioning material of the QTC pressure sensor 150 while being pressed between the fingers 143 of the peristalsis pump 140 and the first tube 131, so as to reduce the wear of the QTC pressure sensor 150 and increase the life thereof. In some embodiments, a material of the wrapping member 160 includes rubber, but the present disclosure is not limited in this regard.

As shown in FIGS. 1 and 3, in the present embodiment, the door assembly 120 includes an outer board 121, an inner board 122, and a cushioning member 123. The inner board 122 is connected to the outer board 121. The cushioning member 123 is connected between the inner board 122 and the outer board 121. With the structural configuration, the force applied to the first tube 131 by the fingers 143 of the peristalsis pump 140 is directly transmitted to the inner board 122 of the door assembly 120 and partially absorbed by the cushioning member 123. Hence, the force applied to the first tube 131 by the fingers 143 of the peristalsis pump 140 can be prevented from directly transmitting to the outer board 121 of the door assembly 120, and thus the outer board 121 can be prevented from being pushed to open the opening 111 of the housing 110. For example, during the pressing operation of the fingers 143 of the peristalsis pump 140 applied to the first tube 131, the inner board 122 will be pushed by the first tube 131 to act similar to a seesaw relative to the outer board 121 through the cushioning member 123.

In some embodiments, the cushioning member 123 of the door assembly 120 is a spring, but the present disclosure is not limited in this regard.

In addition, as shown in FIGS. 1 and 3, in the present embodiment, the infusion tube assembly 130 further includes a fixture 132 and a second tube 133. The fixture 132 is detachably connected to the housing 110 (e.g., in an engaging manner) and configured to clamp an end of the first tube 131. The fixture 132 is further configured to clamp an end of the second tube 133, so as to connect the first tube 131 to the second tube 133.

In some embodiments, an elasticity coefficient of the first tube 131 is greater than an elasticity coefficient of the second tube 133. Therefore, after being pressed by the fingers 143 of the peristalsis pump 140 for a long time, the first tube 131 has a good restoring capability and a long service life. When the first tube 131 reaches its service life, the user can open the fixture 132 and replace a new one of the first tube 131, and the infusion tube assembly 130 can be reused to provide the function of infusion.

In some embodiments, a material of the first tube 131 includes silicone, but the present disclosure is not limited in this regard. In some embodiments, a material of the second tube 133 includes PVC (PolyVinyl Chloride), but the present disclosure is not limited in this regard.

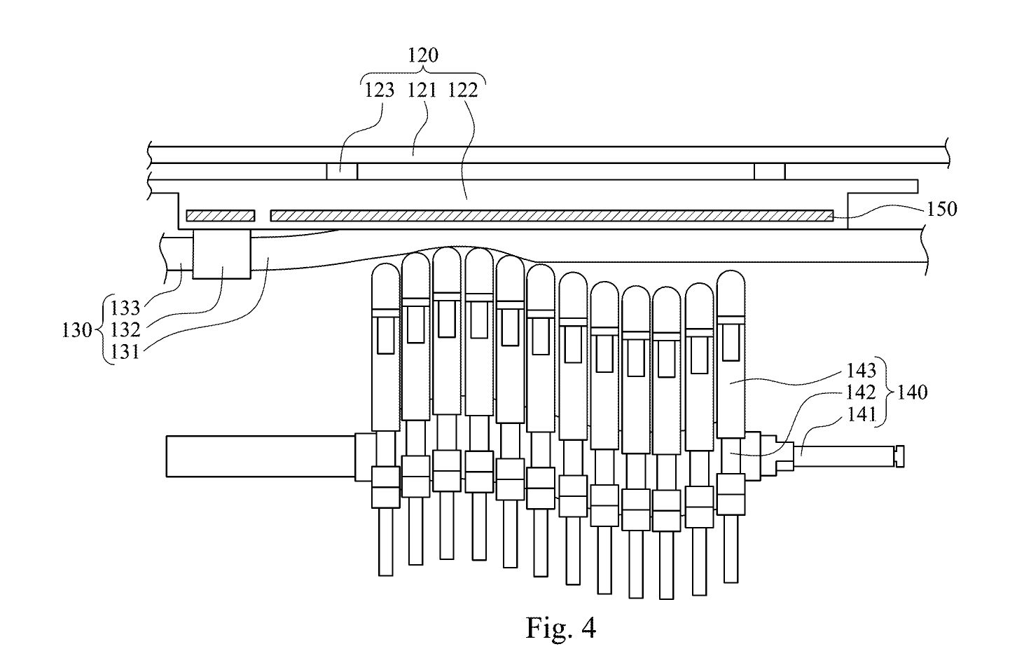

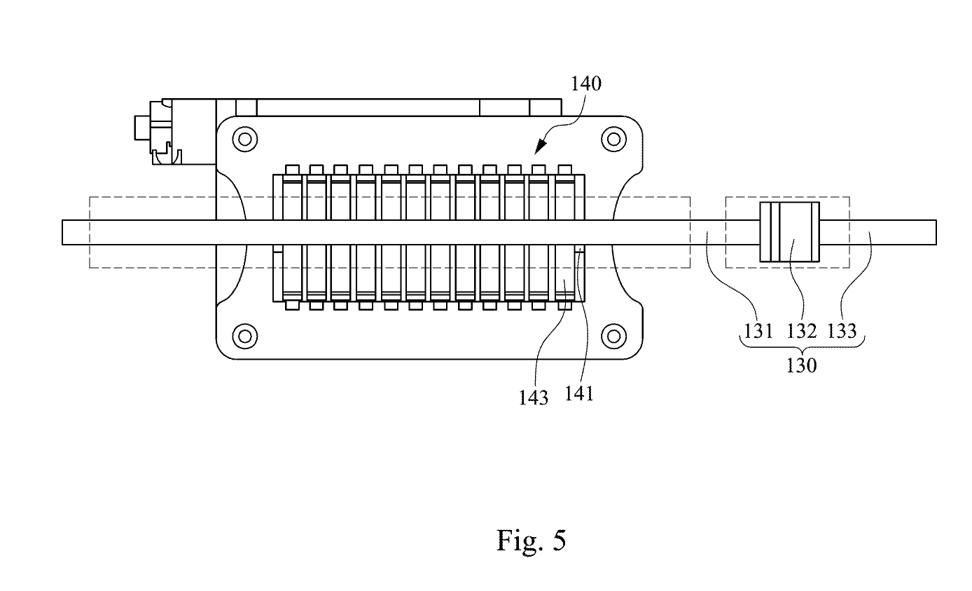

Reference is made to FIGS. 4 and 5. FIG. 4 is a top view of certain components of an infusion apparatus 100 according to another embodiment of the disclosure, in which a door assembly 220 is in a closed state. FIG. 5 is a front view of certain components of an infusion apparatus 100 according to an embodiment of the disclosure. As shown in FIGS. 4 and 5, a difference between the present embodiment and the embodiment shown in FIG. 3 is that the QTC pressure sensor 150 is disposed at the inner board 222 in the present embodiment. Specifically, the QTC pressure sensor 150 is disposed in the inner board 222 of the door assembly 220. When the door assembly 220 covers the opening 111 of the housing 110, the fingers 143 of the peristalsis pump 140 abut against a side of the first tube 131, and the QTC pressure sensor 150 abuts against another side of the first tube 131 through a part of the inner board 222 and is configured to detect pressing operations of the fingers 143 of the peristalsis pump 140 applied to the first tube 131 through the part of the inner board 222.

In some embodiments, the inner board 222 is made of a soft material (e.g., plastics). Therefore, the pressing force of the fingers 143 of the peristalsis pump 140 applied to the first tube 131 can be transmitted to the QTC pressure sensor 150 embedded in the inner board 222 via the inner board 222.

In some embodiments, the QTC pressure sensor 150 is embedded in the inner board 222 of the door assembly 220 by an injection molding process, but the present disclosure is not limited in this regard.

In some embodiments, the outer board 121 of the door assembly 220 can also be made of plastics, and a hardness of the inner board 222 is smaller than a hardness of the outer board 121. In some embodiments, the outer board 121 and the outer board 121 of the door assembly 220 are manufactured by a twin-shot injection molding process, but the present disclosure is not limited in this regard.

In some embodiments, the QTC pressure sensor 150 can be wrapped by the wrapping member 160 shown in FIG. 3 and abut between an outer surface of the inner board 222 and the first tube 131.

As shown in FIG. 5, the location at which the QTC pressure sensor 150 is disposed is indicated by the dashed boxes, and the location not only covers the fingers 143 of the peristalsis pump 140 but also covers the upstream and the downstream of the first tube 131. Therefore, when the QTC pressure sensor 150 detects that the pressure at the upstream of the first tube 131 is smaller than a predetermined value, it can be immediately known that an inadequate infusion phenomenon is occurred at the upstream of the first tube 131 (It might be because the liquid in the infusion bag is depleted or the piping between the first tube 131 and the infusion bag is leaking); and when the QTC pressure sensor 150 detects that the pressure at the downstream of the first tube 131 is greater than a predetermined value, it can be immediately known that an Infusion clogging phenomenon is occurred at the downstream of the first tube 131 (It might be because the second tube 133 of the infusion tube assembly 130 is pressed).

As shown in FIG. 5, the location at which the QTC pressure sensor 150 can further cover the fixture 132 of the infusion tube assembly 130. When the door assembly 220 covers the opening 111 of the housing 110, the inner board 222 of the door assembly 220 abuts against the fixture 132. Therefore, when the door assembly 220 covers the opening 111 of the housing 110, the QTC pressure sensor 150 is further configured to detect an abutting operation of the inner board 222 of the door assembly 220 applied to the fixture 132, so that it can be immediately known whether the fixture 132 is correctly installed in the housing 110 or whether the fixture 132 installed in the housing 110 meets specific specifications.

According to the foregoing recitations of the embodiments of the disclosure, it can be seen that the infusion apparatus of the present disclosure use the QTC pressure sensor to precisely detect the exact two-dimensional position and force value of the point of pressing performed to the infusion tube assembly by the pressing mechanism, so as to become a more accurate closed loop. Therefore, the infusion apparatus of the present disclosure can replace the components known to detect the position of force point (e.g., encoders) and the components known to detect force values (e.g., pressure sensors), so as to achieve the purposes of simplifying the complexities of mechanisms and assembling processes of the infusion apparatus and reduce the overall volume and costs.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. An infusion apparatus, comprising:
a housing having an opening;
a door assembly connected to the housing and configured to cover the opening;
an infusion tube assembly having a first tube located in the housing;
a pressing mechanism disposed in the housing and configured to press the first tube; and
a QTC pressure sensor, wherein when the door assembly covers the opening, the QTC pressure sensor is located between the housing and the door assembly and configured to detect a pressing operation of the pressing mechanism applied to the first tube.

2. The infusion apparatus of claim 1, wherein the pressing mechanism is configured to press the first tube towards the door assembly.

3. The infusion apparatus of claim 2, wherein the QTC pressure sensor is disposed between the pressing mechanism and the first tube.

4. The infusion apparatus of claim 3, further comprising a wrapping member wrapping at an exterior of the QTC pressure sensor.

5. The infusion apparatus of claim 2, wherein the QTC pressure sensor is disposed at the door assembly.

6. The infusion apparatus of claim 5, wherein the door assembly comprises:
an outer board; and
an inner board connected to the outer board, wherein the QTC pressure sensor is disposed at the inner board.

7. The infusion apparatus of claim 6, wherein the QTC pressure sensor is embedded in the inner board.

8. The infusion apparatus of claim 6, wherein a hardness of the inner board is smaller than a hardness of the outer board.

9. The infusion apparatus of claim 6, wherein the door assembly further comprises a cushioning member, and the inner board is connected to the outer board through the cushioning member.

10. The infusion apparatus of claim 1, wherein the infusion tube assembly further comprises a fixture detachably connected to the housing and configured to clamp an end of the first tube, and when the door assembly covers the opening, the door assembly abuts against the fixture, and the QTC pressure sensor is further configured to detect an abutting operation of the door assembly applied to the fixture.

11. The infusion apparatus of claim 10, wherein the infusion tube assembly further comprises a second tube, and the fixture is further configured to clamp an end of the second tube, so as to connect the first tube to the second tube.

12. The infusion apparatus of claim 1, wherein the pressing mechanism is a peristalsis pump.
